(19) United States
(12) Patent Application Publication
(10) Pub. No.: US 2017/0079617 A1
(43) Pub. Date: Mar. 23, 2017

(54) DIAGNOSTIC IMAGING CATHETER

(71) Applicant: TERUMO KABUSHIKI KAISHA, Tokyo (JP)

(72) Inventor: Keiichiro YAMAMOTO, Hiratsuka-city (JP)

(73) Assignee: TERUMO KABUSHIKI KAISHA, Tokyo (JP)

(21) Appl. No.: 15/265,124

(22) Filed: Sep. 14, 2016

(30) Foreign Application Priority Data
Sep. 18, 2015 (JP) 2015-185909

(51) Int. Cl. A61B 8/00; A61B 8/12; A61B 6/00; A61B 8/08

(52) U.S. Cl. CPC A61B 8/445; A61B 8/0891; A61B 8/12; A61B 8/4494; A61B 6/481

(57) ABSTRACT

A diagnostic imaging catheter is disclosed, which includes a sheath that is inserted into a body-cavity in a living body, an ultrasound transducer that is inserted into the sheath and is able to transmit and receive an ultrasound wave, a housing that accommodates the ultrasound transducer, and a drive shaft that includes the housing at the distal end and is rotatably provided inside the sheath. A first region A1 positioned on the distal side closer than the ultrasound transducer in the housing and a second region A2 positioned on the proximal side closer than the ultrasound transducer in the housing are formed so as to have X-ray contrast properties higher than that of an intermediate region A3 positioned between the first region and the second region.

### CROSS-REFERENCES TO RELATED APPLICATIONS

This application claims priority to Japanese Application No. 2015-185909 filed on Sep. 18, 2015, the entire content of which is incorporated herein by reference.

### TECHNICAL FIELD

The present disclosure relates to a diagnostic imaging catheter.

### BACKGROUND DISCUSSION

In the related art, as diagnostic imaging catheters obtaining tomographic images of a blood vessel, there have been catheters which obtain images through an intra-vascular ultrasound (IVUS) diagnostic method.

The diagnostic imaging catheter used in the intra-vascular ultrasound diagnostic method has a configuration in which radial scanning is performed inside a blood vessel by using a probe internally equipped with an ultrasound transducer, processing such as amplification and wave detection is performed after a reflected wave (an ultrasound wave echo) reflected by biological tissue inside a body-cavity is received by the ultrasound transducer, and a cross-sectional image (a diagnostic image) of the blood vessel is depicted based on intensity of a generated ultrasound wave echo (refer to JP-A-2015-119994).

Generally, the diagnostic imaging catheter used in the intra-vascular ultrasound diagnostic method is provided with a radiopaque portion in order to grasp the position of the ultrasound transducer under a radioscopic condition. An operator moves the ultrasound transducer to a target site inside a blood vessel while checking the position of a contrast-imaged radiopaque portion in an X-ray image of a living body captured by an X-ray imaging apparatus, thereby depicting a cross-sectional image of the blood vessel.

In the related art, such a radiopaque portion is formed by causing a housing which accommodates an ultrasound transducer to be radiopaque in its entirety or by attaching a radiopaque member to the distal end or the proximal end of the housing. In the former case, in order to be able to store the ultrasound transducer, the housing is configured to be greater than the ultrasound transducer. Therefore, a wide range including the ultrasound transducer is contrast-imaged, and an operator cannot easily grasp the accurate position of the ultrasound transducer. In the latter case, the position on the distal end or the proximal side deviated from the ultrasound transducer is contrast-imaged. Therefore, an operator needs to perform positioning of the ultrasound transducer in consideration that the ultrasound transducer is located on the proximal side closer than the radiopaque portion. Accordingly, it is desirable to further improve the operability by causing the position of the ultrasound transducer to be easily grasped and causing the ultrasound transducer to be promptly disposed at a predetermined position.

### SUMMARY

The present disclosure has been made in consideration of the aforementioned problem, and provides a diagnostic imaging catheter in which the position of an ultrasound transducer inserted into a living body can be easily grasped in an X-ray image and of which operability is further improved.

A diagnostic imaging catheter is disclosed, which includes a sheath that is inserted into a body-cavity in a living body, an ultrasound transducer that is inserted into the sheath and is able to transmit and receive an ultrasound wave, a housing that accommodates the ultrasound transducer, and a drive shaft that includes the housing at the distal end and is rotatably provided inside the sheath. A first region positioned on the distal side closer than the ultrasound transducer in the housing and a second region positioned on the proximal side closer than the ultrasound transducer in the housing are formed so as to have X-ray contrast properties higher than that of an intermediate region positioned between the first region and the second region.

A diagnostic imaging catheter is disclosed, which includes a sheath; an ultrasound transducer that is inserted into the sheath and configured to transmit and receive an ultrasound wave; a housing that accommodates the ultrasound transducer; and wherein a first region positioned on the distal side closer than the ultrasound transducer in the housing and a second region positioned on the proximal side closer than the ultrasound transducer in the housing are formed so as to have X-ray contrast properties higher than that of an intermediate region positioned between the first region and the second region.

A method is disclosed comprising: introducing a diagnostic imaging catheter into a blood vessel of a human body, the diagnostic imaging catheter including a sheath that is inserted into a body-cavity in a living body, an ultrasound transducer that is inserted into the sheath and configured to transmit and receive an ultrasound wave, a housing that accommodates the ultrasound transducer, and a drive shaft that includes the housing at the distal end and is rotatably provided inside the sheath, wherein a first region positioned on the distal side closer than the ultrasound transducer in the housing and a second region positioned on the proximal side closer than the ultrasound transducer in the housing are formed so as to have X-ray contrast properties higher than that of an intermediate region positioned between the first region and the second region; obtaining an X-ray image of the first and second regions of the diagnostic imaging catheter; and moving the ultrasound transducer to a target site inside the blood vessel based on the X-ray image of the first and second regions.

According to the diagnostic imaging catheter having the above-described configuration, the first region and the second region in the housing are formed so as to have X-ray contrast properties higher than that of the intermediate region where the ultrasound transducer is disposed. Therefore, the position of the ultrasound transducer can be easily grasped by visually checking the difference in gradation of color between the regions of the first region and the second region, and the intermediate region in an X-ray image. As a result thereof, the ultrasound transducer can be accurately and promptly positioned at a desired position inside a biological lumen. Therefore, a diagnostic imaging catheter of which operability is further improved can be provided.

### BRIEF DESCRIPTION OF THE DRAWINGS

FIG. 1 is a plan view schematically illustrating a diagnostic imaging catheter, according to an embodiment of the present invention.

FIGS. 2A and 2B are views schematically illustrating an overall configuration of the diagnostic imaging catheter, according to the embodiment, wherein FIG. 2A is a side view of the diagnostic imaging catheter before executing a pull-back operation (an evacuating operation), and FIG. 2B is a side view of the diagnostic imaging catheter when the pull-back operation is executed.

FIGS. 3A and 3B are views illustrating a configuration of each of units in the diagnostic imaging catheter, according to the embodiment, wherein FIG. 3A is an enlarged sectional view illustrating a configuration of the diagnostic imaging catheter on the distal side, and FIG. 3B is an enlarged sectional view illustrating a configuration of the diagnostic imaging catheter on the proximal side (hand-side).

FIG. 4 is an enlarged view illustrating a configuration of a housing of the diagnostic imaging catheter, according to the embodiment.

FIG. 5 is a schematic view illustrating a state of capturing an X-ray image of a living body by using an X-ray imaging apparatus in a state where the diagnostic imaging catheter according to the embodiment is inserted into the living body.

FIGS. 6A-6C are X-ray images of a living body captured by using the X-ray imaging apparatus, wherein FIG. 6A is an image captured in a state where the diagnostic imaging catheter according to the embodiment is inserted into a living body, and FIGS. 6B and 6C are images captured in a state where a diagnostic imaging catheter according to a comparative example is inserted into the living body.

FIG. 7 is an enlarged view illustrating a configuration of a housing of the diagnostic imaging catheter, according to a modification example of the embodiment.

### DETAILED DESCRIPTION

Hereinafter, with reference to the accompanying drawings, an embodiment of the present invention will be described. Note that, the description below does not limit the meanings of the technical scope and the terms disclosed in Claims. In addition, for the convenience of description, there are cases where the dimensional ratios of the drawings are exaggerated and are different from the actual ratios.

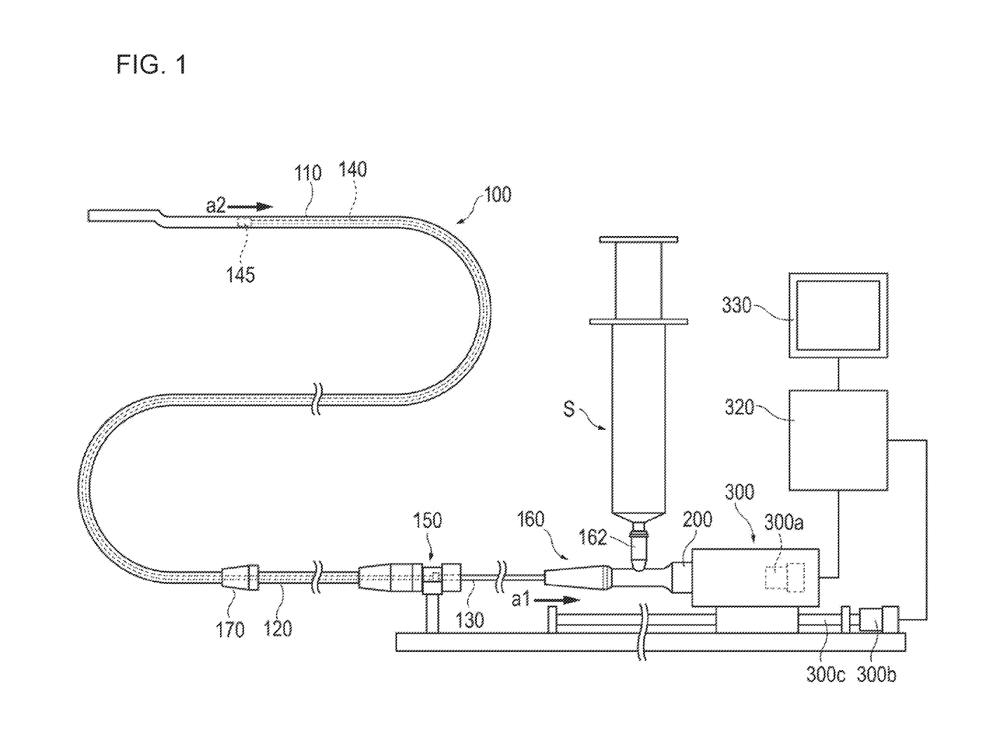

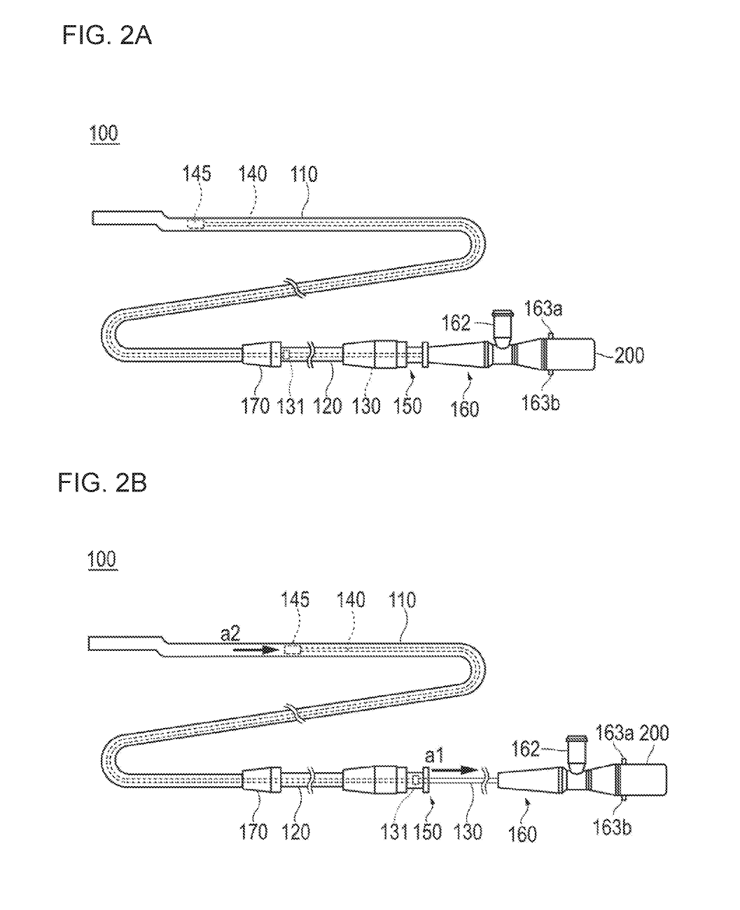

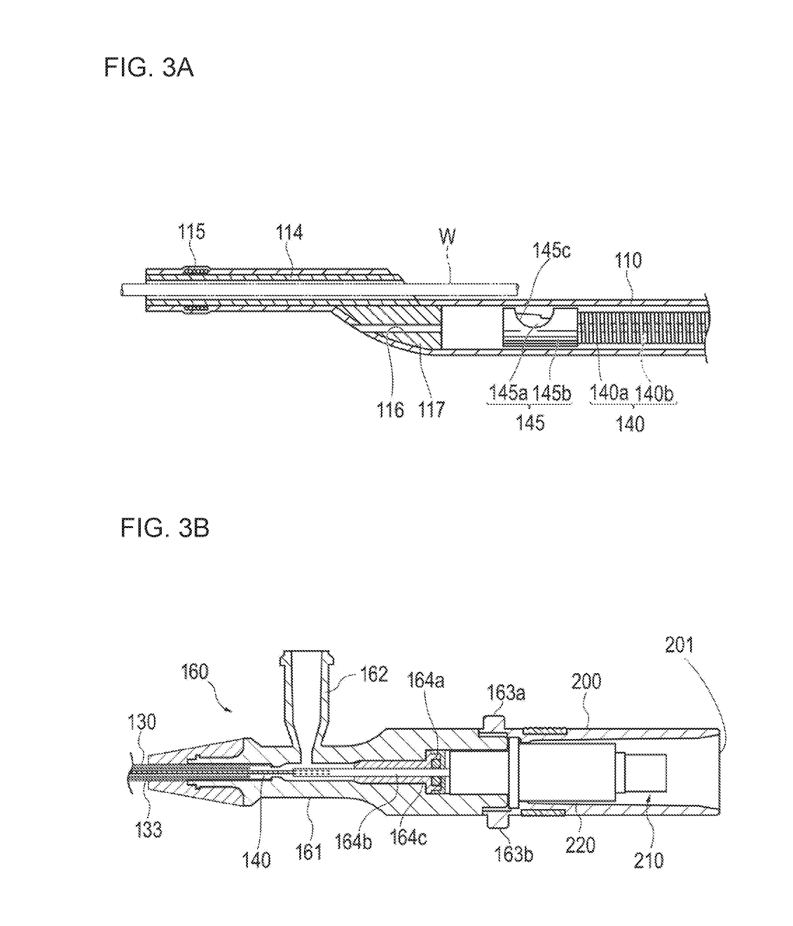

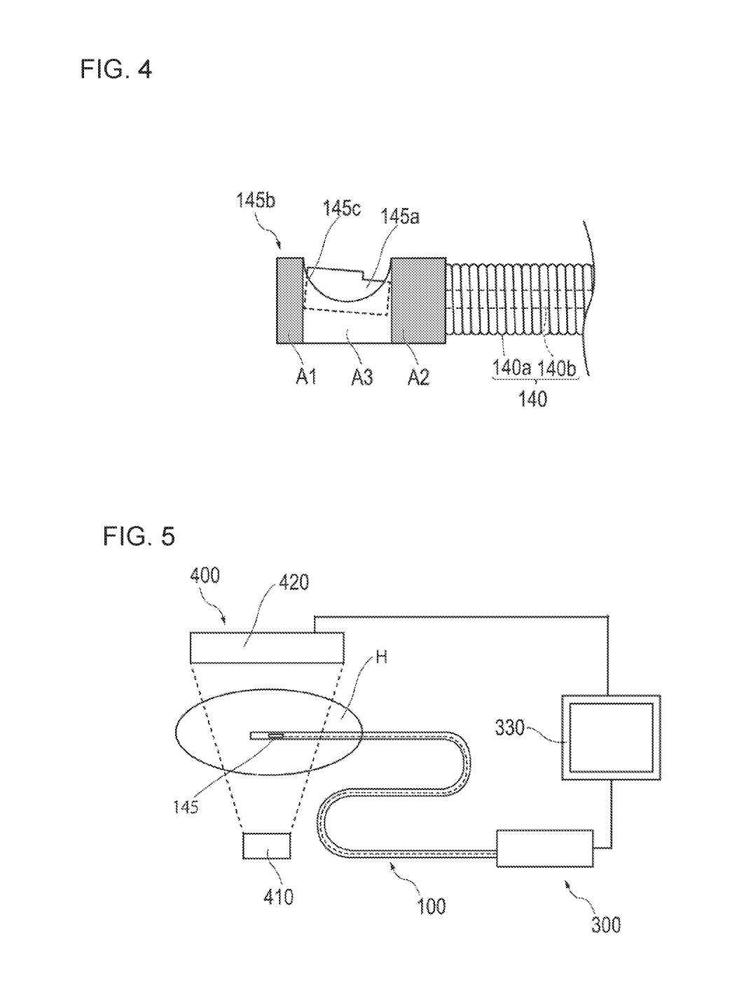

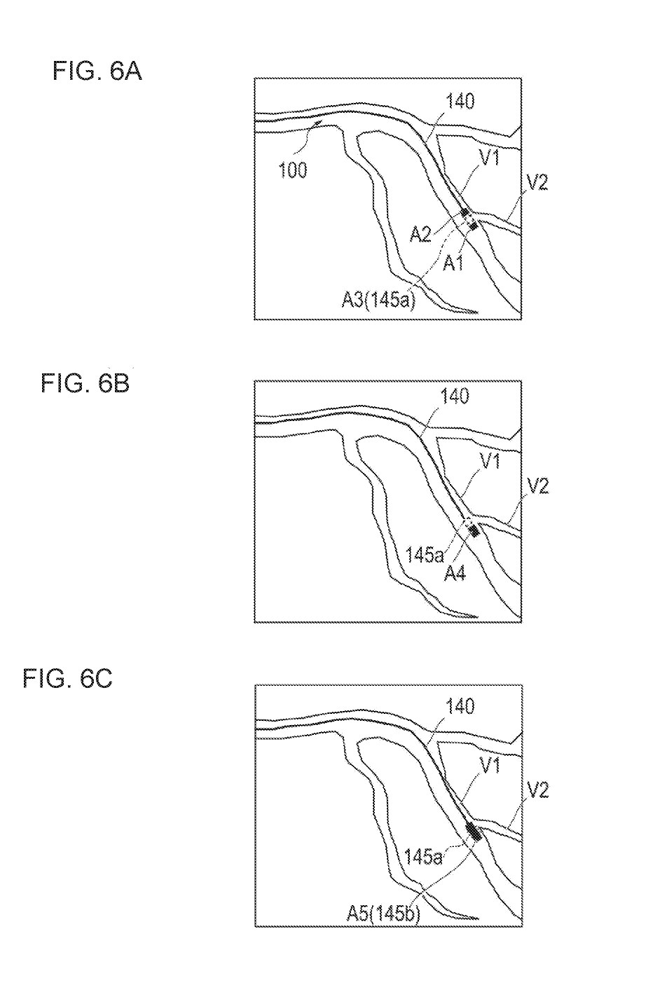

FIG. 1 is a plan view illustrating a state where an external apparatus 300 is connected to a diagnostic imaging catheter 100, according to the embodiment. FIGS. 2A and 2B are views schematically illustrating an overall configuration of the diagnostic imaging catheter 100, according to the embodiment. FIGS. 3A and 3B are views illustrating a configuration of each of units in the diagnostic imaging catheter 100, according to the embodiment. FIG. 4 is an enlarged view illustrating a configuration of a housing 145b of the diagnostic imaging catheter 100, according to the embodiment. FIG. 5 is a schematic view illustrating a state of capturing an X-ray image of a living body H by using an X-ray imaging apparatus 400 in a state where the diagnostic imaging catheter 100 according to the embodiment is inserted into the living body H. FIG. 6 is an X-ray image of a living body captured by using the X-ray imaging apparatus 400. Note that, in FIG. 6, an ultrasound transducer 145a is not clearly contrast-imaged in the X-ray image. However, in order to make the image easy to be understood, the position of the ultrasound transducer 145a is indicated with a dotted line.

The diagnostic imaging catheter 100 according to the present embodiment is a diagnostic imaging catheter which is used in an intra-vascular ultrasound diagnostic method. As illustrated in FIG. 1, the diagnostic imaging catheter 100 is driven by being connected to the external apparatus 300. In addition, as illustrated in FIG. 5, when the diagnostic imaging catheter 100 inserted into a living body is operated, the X-ray imaging apparatus 400 for capturing an X-ray image is used. Hereinafter, each of the apparatuses will be described in detail.

With reference to FIGS. 1 to 4, the diagnostic imaging catheter 100 will be described.

As illustrated in FIGS. 1, 2A, and 2B, generally, the diagnostic imaging catheter 100 can include a sheath 110 that is inserted into a body-cavity in a living body, an outer tube 120 that is provided on the proximal side of the sheath 110, an inner shaft 130 that is inserted into the outer tube 120 so as to be movable forward and rearward, a drive shaft 140 that has a signal transmitting and receiving unit 145 transmitting and receiving a signal at the distal end and is rotatably provided inside the sheath 110, a unit connector 150 that is configured to be provided on the proximal side of the outer tube 120 and to accept the inner shaft 130, and a hub 160 that is provided on the proximal side of the inner shaft 130.

In description of the specification, a side of the diagnostic imaging catheter 100 inserted into a body-cavity will be referred to as the distal end or the distal side, a side of the hub 160 provided in the diagnostic imaging catheter 100 will be referred to as the proximal end or the proximal side, and the extending direction of the sheath 110 will be referred to as the axial direction.

As illustrated in FIGS. 2A and 3B, the drive shaft 140 passes through the sheath 110, the outer tube 120 connected to the proximal end of the sheath 110, and the inner shaft 130 inserted into the outer tube 120, and extends to the inside of the hub 160.

The hub 160, the inner shaft 130, the drive shaft 140, and the signal transmitting and receiving unit 145 are connected to each other so as to integrally move forward and rearward in the axial direction. Therefore, for example, when the hub 160 is operated so as to be pushed toward the distal side, the inner shaft 130 connected to the hub 160 is thrust into the outer tube 120 and the unit connector 150, and the drive shaft 140 and the signal transmitting and receiving unit 145 move inside the sheath 110 toward the distal side. For example, when the hub 160 is operated so as to be pulled toward the proximal side, as indicated with the arrow a1 in FIGS. 1 and 2B, the inner shaft 130 is drawn out through the outer tube 120 and the unit connector 150, and as indicated with the arrow a2, the drive shaft 140 and the signal transmitting and receiving unit 145 move inside the sheath 110 toward the proximal side.

As illustrated in FIG. 2A, when the inner shaft 130 is thrust toward the distal side to the end, the distal portion of the inner shaft 130 reaches the vicinity of a relay connector 170. In this case, the signal transmitting and receiving unit 145 is positioned in the vicinity of the distal end of the sheath 110. The relay connector 170 is a connector which causes the sheath 110 and the outer tube 120 to be connected to each other.

As illustrated in FIG. 2B, a coming-off prevention connector 131 is provided at the distal end of the inner shaft 130. The coming-off prevention connector 131 has a function of preventing the inner shaft 130 from coming off from the outer tube 120. The coming-off prevention connector 131 is configured to be caught at a predetermined position in the inner wall of the unit connector 150 when the hub 160 is pulled toward the proximal side to the end, for example, when the inner shaft 130 is drawn out to the end through the outer tube 120 and the unit connector 150. Note that, in order to prevent the inner shaft 130 from coming off from the outer tube 120, the coming-off prevention connector 131 is not necessarily provided. For example, the inner shaft 130 may be prevented from coming off from the outer tube 120 by processing the distal end of the inner shaft 130 such that the inner shaft 130 does not come off from the outer tube 120.

As illustrated in FIG. 3A, the drive shaft 140 can include an elastic pipe body 140a and a signal wire 140b which is inserted through the inside of the pipe body 140a. For example, the pipe body 140a can be configured to be a coil having multiple layers of which winding directions around the axis are different from each other. As a configuration material of the coil, for example, stainless steel and a nickel-titanium (Ni—Ti) alloy can be used. For example, the signal wire 140b can be configured to be a twist pair cable or a coaxial cable.

The signal transmitting and receiving unit 145 has an ultrasound transducer 145a transmitting and receiving an ultrasound wave, and a housing 145b in which the ultrasound transducer 145a is housed.

The ultrasound transducer 145a has a function of transmitting an ultrasound wave as an inspection wave into a body-cavity and receiving the ultrasound wave reflected from the body-cavity. The ultrasound transducer 145a is electrically connected to the below-described electrode terminal 210 via the signal wire 140b.

As the ultrasound transducer 145a, for example, a piezoelectric material such as ceramics and crystal can be adopted.

The housing 145b is formed to have a tubular shape, and the proximal side of the housing 145b is fixed to the drive shaft 140. The method of fixing the housing 145b and the drive shaft 140 is not particularly limited. For example, the housing 145b and the drive shaft 140 can be glued together by using an adhesive or performing soldering.

In addition, the ultrasound transducer 145a is stored inside the housing 145b. A portion facing the ultrasound wave transmitting and receiving unit of the ultrasound transducer 145a in the housing 145b is notched, thereby forming an opening portion 145c.

In this manner, the housing 145b needs to internally store the ultrasound transducer 145a and the proximal side of the housing 145b needs to be fixed to the drive shaft 140. Therefore, the length of the housing 145b in the axial direction becomes greater than the length of the ultrasound transducer 145a in the axial direction (refer to FIG. 4). For example, when the length of the ultrasound transducer 145a in the axial direction ranges from, for example, 0.5 to 1.0 mm, the length of the housing 145b in the axial direction ranges from approximately 1.5 to 2 mm.

As illustrated in FIG. 4, in this specification, a region positioned on the distal side closer than the ultrasound transducer 145a in the housing 145b will be referred to as “a first region A1”, a region positioned on the proximal side closer than the ultrasound transducer 145a in the housing 145b will be referred to as “a second region A2”, and a region between the first region A1 and the second region A2 will be referred to as “an intermediate region A3”.

The first region A1 and the second region A2 in the housing 145b are formed so as to have X-ray contrast properties higher than that of the intermediate region A3 under a radioscopic condition, for example, such that the intermediate region A3 can have X-ray transmissivity lower than that of the first and second regions A1, A2. In the present embodiment, the housing 145b is formed of a material having X-ray transmittance. In addition, the first region A1 and the second region A2 in the housing are coated with a radiopaque material including high contrast properties as high as the boundaries with respect to the intermediate region A3 can be clearly and visually recognized under a radioscopic condition (in the diagram, the coated place is high-lighted in grey). For example, in a case where SUS is adopted as a radioparent material, platinum, gold, iridium, and the like can be adopted as a radiopaque material. Accordingly, when an image of the housing 145b is captured by using the below-described X-ray imaging apparatus 400, the first region A1 and the second region A2 are contrast-imaged as dark as the boundaries with respect to the intermediate region A3 can be clearly and visually recognized. For example, in the example illustrated in FIG. 6A, the position of the ultrasound transducer 145a in the X-ray image can be easily grasped as a white portion interposed between the portions of the first region A1 and the second region A2 which are contrast-imaged in dark color.

Note that, FIG. 4 illustrates an example of a case where the entire regions of the first region A1 and the second region A2 are coated with a material having radiopaque properties. However, there is no need for the entire regions of the first region A1 and the second region A2 to be coated as long as the boundaries between regions A1 and A2 and the intermediate region A3 can be visually recognized in the X-ray image, which is enough. For example, a portion of the first region A1 on the proximal side and a portion of the second region A2 on the distal side may be coated with a material having radiopaque properties.

As illustrated in FIG. 3A, a priming liquid discharge member 117 having a priming liquid discharge hole 116 for discharging priming liquid formed therein is installed at the distal portion of the sheath 110. When the diagnostic imaging catheter 100 is in use, in order to reduce attenuation of ultrasound waves caused by air inside the sheath 110 and to efficiently transmit and receive the ultrasound waves, the inside of the sheath 110 is filled with the priming liquid. When the sheath 110 is filled with the priming liquid, gas such as air staying inside the sheath 110 can be discharged through the priming liquid discharge hole 116 formed in the priming liquid discharge member 117.

In accordance with an exemplary embodiment, the sheath 110 can be formed of a material having high transmittance of an ultrasound wave. A range in which the ultrasound transducer 145a moves in the axial direction of the sheath 110 (the distal portion of the sheath 110) is configured to be an acoustic window portion of which transmittance of an ultrasound wave is formed to be higher than other portions.

The sheath 110 is formed of an elastic material, and the material is not particularly limited. For example, the sheath 110 can be made from various types of thermoplastic elastomers such as a styrene-based elastomer, a polyolefin-based elastomer, a polyurethane-based elastomer, a polyester-based elastomer, a polyimide-based elastomer, a polyimide-based elastomer, a polybutadiene-based elastomer, a trans-polyisoprene-based elastomer, a fluororubber-based elastomer, and a chlorinated polyethylene-based elastomer. One type or a combination of two or more types among thereof (a polymer alloy, a polymer blend and a laminated body) can also be adopted. Note that, a hydrophilic lubrication coating layer exhibiting lubricity at the time of wetting can be disposed on the outer surface of the sheath 110.

A guide wire insertion member 114 provided with a lumen through which a guide wire W can be inserted is attached to the distal portion of the sheath 110. In addition, the guide wire insertion member 114 is provided with a marker 115 having X-ray contrast properties.

As illustrated in FIG. 3B, the hub 160 has a hollow-shaped hub main body 161, a connector unit 200 in which the electrode terminal 210 mechanically and electrically connected to the below-described external apparatus 300 is disposed, a port 162 which communicates with the inside of the hub main body 161, direction checking spurs 163a and 163b for checking the orientation of the hub 160 when performing connection with respect to the external apparatus 300, a seal member 164a which seals a place on the proximal side closer than the port 162, a connection pipe 164b which holds the drive shaft 140, and a bearing 164c which rotatably supports the connection pipe 164b.

The inner shaft 130 is connected to the distal portion of the hub main body 161. The drive shaft 140 is drawn out through the inner shaft 130 inside the hub main body 161. A protection tube 133 is disposed between the inner shaft 130 and the drive shaft 140. The protection tube 133 has a function of pressing vibration (flapping) of the drive shaft 140 caused by the clearance generated at the time of pulling-back.

In order to transfer rotations of the rotor 220 to the drive shaft 140, the connection pipe 164b holds the drive shaft 140 at the end portion on a side opposite to the rotor 220 (the distal end of the connection pipe 164b). The signal wire 140b (refer to FIG. 3A) is inserted through the inside of the connection pipe 164b. One end of the signal wire 140b is connected to the electrode terminal 210, and the other end passes through the inside of the drive shaft 140 and is connected to the ultrasound transducer 145a. A signal received by the ultrasound transducer 145a is transmitted to the external apparatus 300 via the electrode terminal 210 and is subjected to predetermined processing, thereby being displayed as an image.

Repeatedly with reference to FIG. 1, the diagnostic imaging catheter 100 is driven by being connected to the external apparatus 300.

As described above, the external apparatus 300 is connected to the connector unit 200 provided on the proximal side of the hub 160.

In addition, the external apparatus 300 has a motor 300a which is a power source for rotating the drive shaft 140, and a motor 300b which is a power source for moving the drive shaft 140 in the axial direction. A rotary motion of the motor 300b is converted into a motion in the axial direction by a ball screw 300c connected to the motor 300b.

An operation of the external apparatus 300 is controlled by a control apparatus 320 which is electrically connected thereto. The control apparatus 320 includes a central processing unit (CPU) and a memory as main configuration elements. The control apparatus 320 is electrically connected to a monitor 330.

With reference to FIG. 5, when the diagnostic imaging catheter 100 inserted into the living body H is operated, the X-ray imaging apparatus 400 for capturing an X-ray image is adopted.

The X-ray imaging apparatus 400 has an X-ray source 410 generating an X-ray, an X-ray detector 420 detecting an X-ray, and the monitor 330 displaying an X-ray image obtained through the X-ray detector 420. In the present embodiment, the X-ray imaging apparatus 400 shares the monitor 330 with the external apparatus 300.

The X-ray source 410 and the X-ray detector 420 are disposed so as to interpose the living body H in which the diagnostic imaging catheter 100 is inserted. When an X-ray is generated from the X-ray source 410, the X-ray transmitted through the living body H and the diagnostic imaging catheter 100 is detected by the X-ray detector 420. The monitor 330 is electrically connected to the X-ray detector 420 and displays the obtained X-ray image.

Subsequently, an example of use of the diagnostic imaging catheter 100, the external apparatus 300, and the X-ray imaging apparatus 400 will be described.

First, as illustrated in FIG. 1, the external apparatus 300 is connected to the connector unit 200 of the diagnostic imaging catheter 100. Thereafter, a user connects a syringe S having the priming liquid therein to the port 162 and fills the sheath 110 with the priming liquid by pressing a plunger of the syringe S.

After performing priming, as illustrated in FIG. 2A, the user thrusts the hub 160 until the hub 160 abuts on the proximal end of the unit connector 150 and moves the signal transmitting and receiving unit 145 to the distal side. In this state, the sheath 110 is inserted into a body-cavity (for example, a blood vessel) along the guide wire W toward a target position. In this case, an operator can deliver the sheath 110 to a target position while checking the position of the marker 115 provided in the sheath 110, in the X-ray image captured by using the X-ray imaging apparatus 400.

When a tomographic image is obtained at a target position inside a body-cavity, as illustrated in FIG. 2B, the signal transmitting and receiving unit 145 transmits and receives an ultrasound wave while moving together with the drive shaft 140 toward the proximal side. In addition, in this case, the signal transmitting and receiving unit 145 rotates together with the drive shaft 140.

The control apparatus 320 controls the motor 300a illustrated in FIG. 1 and controls rotation around the axis of the drive shaft 140. In addition, the control apparatus 320 controls the motor 300b and controls movement of the drive shaft 140 in the axial direction.

The signal transmitting and receiving unit 145 transmits an ultrasound wave to the inside of a body based on a signal sent from the control apparatus 320. A signal received by the signal transmitting and receiving unit 145 and corresponding to a reflected wave is sent to the control apparatus 320 via the drive shaft 140 and the external apparatus 300. The control apparatus 320 generates a tomographic image of a body-cavity based on a signal sent from the signal transmitting and receiving unit 145 and causes the monitor 330 to display the generated image.

The connector unit 200 provided inside the hub 160 rotates in a state of being connected to the external apparatus 300, and the drive shaft 140 rotates in association therewith. The rotating speed of the connector unit 200 and the drive shaft 140 can be 1,800 rpm, for example.

The diagnostic imaging catheter 100 can be used not only in a case where the ultrasound transducer 145a is thrust into a predetermined position in the sheath 110 on the distal side and the ultrasound transducer 145a is automatically moved to the proximal side by the motor as described above, but can also be used in a case where an operator moves the ultrasound transducer 145a to a particular site so as to observe the particular site. For example, as illustrated in FIG. 6A, a blocked state and the like in the vicinity of the entrance of a lateral branch V2 bifurcated from a main duct V1 of a blood vessel can be checked by using the diagnostic imaging catheter 100.

In this case, in the diagnostic imaging catheter 100 according to the present embodiment, the intermediate region A3 may be set so as to be disposed at the entrance of the lateral branch V2 based on the difference in gradation of color between the regions A1 and A2 and the intermediate region A3 in the X-ray image. In this manner, the operator can easily grasp the position of the ultrasound transducer 145a, and thus, the operator can promptly move the ultrasound transducer 145a to the entrance of the lateral branch V2.

Note that, for example, as in the related art, in a case where a member A4 having radiopaque properties is attached to the distal end of the housing 145b, in consideration that the ultrasound transducer 145a is positioned on the proximal side closer than the member A4 having radiopaque properties, as illustrated in FIG. 6B, the operator needs to perform positioning of the ultrasound transducer 145a. In addition, for example, in a case where a member having radiopaque properties is attached to the proximal end of the housing 145b as in the related art (not illustrated), in consideration that the ultrasound transducer 145a is positioned on the distal side closer than the member having radiopaque properties, the operator needs to perform positioning of the ultrasound transducer 145a.

In addition, for example, as in the related art, in a case where the housing 145b in its entirety is configured to be formed of a radiopaque material, since the housing 145b is greater than the ultrasound transducer 145a, as illustrated in FIG. 6C, a wide range A5 including the ultrasound transducer 145a is contrast-imaged. Therefore, the operator cannot easily grasp the position of the ultrasound transducer 145a, and there are cases where even though the contrast-imaged range A5 is aligned with the entrance of the lateral branch V2, the ultrasound transducer 145a is disposed at a position deviated from the entrance of the lateral branch V2. Even though depiction of a cross-sectional image is performed by using the diagnostic imaging catheter 100 in this state, the ultrasound transducer 145a depicts a cross-sectional image of a position deviated from the entrance of the lateral branch V2. Therefore, the operator needs to additionally perform a fine adjustment of the position of the ultrasound transducer 145a.

In accordance with an exemplary embodiment, in the present embodiment, as a paradigm of prompt positioning of the ultrasound transducer 145a performed based on the first region A1 and the second region A2, description has been given regarding a case where the vicinity of the entrance of the lateral branch V2 is intended to be observed. In addition thereto, for example, when the prompt positioning of the ultrasound transducer 145a performed based on the first region A1 and the second region A2 is utilized, the ultrasound transducer 145a can be accurately disposed at a site where a stent is scheduled to be disposed (a stenosed site), and thus, a stent having more optimal dimensions can be selected based on the depicted cross-sectional image.

As described above, the diagnostic imaging catheter 100 according to the present embodiment includes the sheath 110 that is inserted into a body-cavity in a living body, the ultrasound transducer 145a that is inserted into the sheath 110 and is able to transmit and receive an ultrasound wave, the housing 145b that accommodates the ultrasound transducer 145a, and the drive shaft 140 that includes the housing 145b at the distal end and is rotatably provided inside the sheath 110. The first region A1 positioned on the distal side closer than the ultrasound transducer 145a in the housing 145b and the second region A2 positioned on the proximal side closer than the ultrasound transducer 145a in the housing 145b are formed so as to have X-ray contrast properties higher than that of the intermediate region A3 positioned between the first region A1 and the second region A2.

According to the diagnostic imaging catheter 100 having such a configuration, the first region A1 and the second region A2 in the housing 145b are formed so as to have X-ray contrast properties higher than that of the intermediate region A3 where the ultrasound transducer 145a is disposed. Therefore, the position of the ultrasound transducer 145a can be easily grasped by visually checking the difference in gradation of color between the regions of the first region A1 and the second region A2, and the intermediate region A3 in the X-ray image. As a result thereof, the ultrasound transducer 145a can be accurately and promptly positioned at a desired position inside a biological lumen. Therefore, it is possible to provide the diagnostic imaging catheter 100 of which operability is further improved.

In addition, the first region A1 and the second region A2 in the housing 145b have radiopaque properties. An operator can more clearly and visually recognize the boundaries between the intermediate region A3, and the first region A1 and the second region A2 under a radioscopic condition, and thus, the position of the ultrasound transducer 145a can be easily grasped.

In addition, the first region A1 and the second region A2 in the housing 145b are coated with a material having radiopaque properties. In this manner, it can be relatively easy to apply radiopaque properties to the first region A1 and the second region A2 by only coating the housing 145b with a material having radiopaque properties.

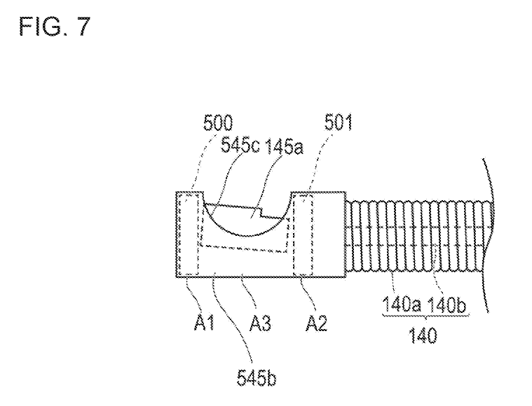

With reference to FIG. 7, a housing 545b according to a modification example of the above-described embodiment will be described. Note that, the same reference numerals and signs will be applied to the configurations similar to those in the above-described embodiment, and description thereof will be omitted.

Similar to the above-described embodiment, the housing 545b according to the modification example is formed to have a tubular shape, and the proximal side of the housing 545b is fixed to the drive shaft 140. In addition, the ultrasound transducer 145a is stored inside the housing 545b. A portion facing the ultrasound wave transmitting and receiving unit of the ultrasound transducer 145a in the housing 545b is notched, thereby forming an opening portion 545c.

The housing 545b is formed of a material having X-ray transmittance, and a first ring 500 and a second ring 501 having radiopaque properties (correspond to “the members having radiopaque properties”) are respectively provided inside the first region A1 and the second region A2 in the housing 545b. Note that, the rings 500 and 501 are provided inside the housing 545b such that end surfaces thereof are respectively disposed at the boundaries between the regions A1 and A2 and the intermediate region A3.

Similar to the above-described embodiment, for example, in a case where SUS is adopted as a material having X-ray transmittance, platinum, gold, and iridium, can be adopted as a material having radiopaque properties, for example. When an image of the housing 545b is captured by using the X-ray imaging apparatus 400, the first region A1 and the second region A2 are contrast-imaged darker than the intermediate region A3 to the extent that the boundaries with respect to the intermediate region A3 can be clearly and visually recognized.

According to the diagnostic imaging catheter 100 of the modification example having such a configuration, the first ring 500 and the second ring 501 having radiopaque properties are respectively provided inside the first region A1 and the second region A2 in the housing 545b. An operator can clearly and visually recognize the boundaries between the intermediate region A3, and the first region A1 and the second region A2 under a radioscopic condition, and thus, the position of the ultrasound transducer 145a can be easily grasped. As a result thereof, the ultrasound transducer 145a can be accurately and promptly positioned at a desired position inside a biological lumen. Therefore, it is possible to provide the diagnostic imaging catheter 100 of which operability is further improved.

Hereinbefore, the diagnostic imaging catheter according to the present invention has been described through the embodiment and the modification example. However, the present invention is not limited to only the configurations described in the embodiment and the modification example and can be suitably changed based on Claims.

For example, as a diagnostic imaging catheter which is a target to be applied with the above-described diagnostic imaging catheter, the diagnostic imaging catheter used in the intra-vascular ultrasound diagnostic method (IVUS) is exemplified. However, the diagnostic imaging catheter which is a target to be applied is not particularly limited as long as the diagnostic imaging catheter is used while an operator checks the position of a sensor in an X-ray image and the sensor is disposed at a desired position. For example, the above-described diagnostic imaging catheter can be applied to a hybrid-type (dual-type) diagnostic imaging catheter which can be used in both the intra-vascular ultrasound diagnostic method and an optical coherence tomography (OCT) diagnostic method.

In addition, the technology of the present disclosure can be applied to a diagnostic imaging catheter which is used in the optical coherence tomography diagnostic method. In this case, a first region positioned on the distal side closer than an optical lens and a second region positioned on the proximal side closer than the optical lens in the housing where the optical lens is accommodated may be configured to be formed so as to have X-ray contrast properties higher than an intermediate region positioned between the first region and the second region.

In addition, in the above-described embodiment, description has been given regarding a case where the first region and the second region in the housing are coated with a material having radiopaque properties, and in the above-described modification example, description has been given regarding a case where the members having radiopaque properties (the first ring and the second ring) are provided inside the first region and the second region in the housing. However, the configuration of applying radiopaque properties to the first region and the second region in the housing is not limited thereto. For example, the first region and the second region in the housing may be configured to be applied with radiopaque properties by causing a material having radiopaque properties to be included in the configuration material of the housing of the first region and the second region. In addition, the first region, the second region, and the intermediate region in the housing may be formed with a member different from each other, and the housing may be formed by causing a member forming the intermediate region having X-ray transmittance to be interposed between members forming the first region and the second region having radiopaque properties and causing the members to be bonded together. In addition, the members having radiopaque properties (the first ring, the second ring, and the like) may be provided outside the first region and the second region in the housing.

The detailed description above describes a diagnostic imaging catheter. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A diagnostic imaging catheter comprising:
a sheath that is inserted into a body-cavity in a living body;
an ultrasound transducer that is inserted into the sheath and configured to transmit and receive an ultrasound wave;
a housing that accommodates the ultrasound transducer; and
a drive shaft that includes the housing at the distal end and is rotatably provided inside the sheath,
wherein a first region positioned on the distal side closer than the ultrasound transducer in the housing and a second region positioned on the proximal side closer than the ultrasound transducer in the housing are formed so as to have X-ray contrast properties higher than that of an intermediate region positioned between the first region and the second region.

2. The diagnostic imaging catheter according to claim 1,
wherein the first region and the second region in the housing have radiopaque properties.

3. The diagnostic imaging catheter according to claim 2,
wherein the first region and the second region in the housing are coated with a material having radiopaque properties.

4. The diagnostic imaging catheter according to claim 1,
wherein the first region and the second region in the housing are coated with a material having radiopaque properties.

5. The diagnostic imaging catheter according to claim 1,
wherein members having radiopaque properties are respectively provided inside the first region and the second region in the housing.

6. The diagnostic imaging catheter according to claim 2,
wherein members having radiopaque properties are respectively provided inside the first region and the second region in the housing.

7. The diagnostic imaging catheter according to claim 4,
wherein members having radiopaque properties are respectively provided inside the first region and the second region in the housing.

8. The diagnostic imaging catheter according to claim 1,
wherein a length of the housing in an axial direction is greater than a length of the ultrasound transducer in the axial direction.

9. The diagnostic imaging catheter according to claim 4,
wherein an entirety of the first region and the second region are not coated with the material having radiopaque properties.

10. The diagnostic imaging catheter according to claim 9,
wherein a portion of the first region on a proximal side and a portion of the second region on a distal are coated with the material having radiopaque properties.

11. A diagnostic imaging catheter comprising:
a sheath;
an ultrasound transducer that is inserted into the sheath and configured to transmit and receive an ultrasound wave;
a housing that accommodates the ultrasound transducer; and
wherein a first region positioned on the distal side closer than the ultrasound transducer in the housing and a second region positioned on the proximal side closer than the ultrasound transducer in the housing are formed so as to have X-ray contrast properties higher than that of an intermediate region positioned between the first region and the second region.

12. The diagnostic imaging catheter according to claim 11,
wherein the first region and the second region in the housing have radiopaque properties.

13. The diagnostic imaging catheter according to claim 12,
wherein the first region and the second region in the housing are coated with a material having radiopaque properties.

14. The diagnostic imaging catheter according to claim 11,
wherein the first region and the second region in the housing are coated with a material having radiopaque properties.

15. The diagnostic imaging catheter according to claim 11,
wherein members having radiopaque properties are respectively provided inside the first region and the second region in the housing.

16. The diagnostic imaging catheter according to claim 12,
wherein members having radiopaque properties are respectively provided inside the first region and the second region in the housing.

17. The diagnostic imaging catheter according to claim 14,
wherein members having radiopaque properties are respectively provided inside the first region and the second region in the housing.

18. A method comprising:
introducing a diagnostic imaging catheter into a blood vessel of a human body, the diagnostic imaging catheter including a sheath that is inserted into a body-cavity in a living body, an ultrasound transducer that is inserted into the sheath and configured to transmit and receive an ultrasound wave, a housing that accommodates the ultrasound transducer, and a drive shaft that includes the housing at the distal end and is rotatably provided inside the sheath, wherein a first region positioned on the distal side closer than the ultrasound transducer in the housing and a second region positioned on the proximal side closer than the ultrasound transducer in the housing are formed so as to have X-ray contrast properties higher than that of an intermediate region positioned between the first region and the second region;
obtaining an X-ray image of the first and second regions of the diagnostic imaging catheter; and
moving the ultrasound transducer to a target site inside the blood vessel based on the X-ray image of the first and second regions.

19. The method according to claim 18, comprising:
setting the intermediate region at an entrance of the lateral branch based on the difference in gradation of color between the first and second region and the intermediate region in the X-ray image.
